(19) United States
(12) Patent Application Publication
(10) Pub. No.: US 2018/0236010 A1
(43) Pub. Date: Aug. 23, 2018

(54) SYSTEMS AND METHODS FOR IMPROVING PRODUCTIVITY, HEALTH AND LIFE EXPECTANCY IN AQUACULTURE INDUSTRY

(71) Applicant: Mahalakshmi N. A. SRINIVASAN, Navi Mumbai (IN)

(72) Inventor: Mahalakshmi N. A. SRINIVASAN, Navi Mumbai (IN)

(21) Appl. No.: 15/551,552

(22) PCT Filed: Sep. 23, 2016
(86) PCT No.: PCT/IB2016/055689

§ 371 Date: Aug. 16, 2017

(30) Foreign Application Priority Data
Jun. 10, 2016 (IN) 201621019900

(51) Int. Cl. A61K 35/74; C02F 3/34; A23K 10/18; A23K 50/80; A01K 61/00

(52) U.S. Cl. CPC A61K 35/74; C02F 3/34; C02F 2103/20; A23K 50/80; A01K 61/00; A23K 10/18

(57) ABSTRACT

A system and method, to improve productivity, health and Life expectancy of aquatic animals using an efficient and cost effective natural effector composition comprised of gram negative bacilli and is available in suitable packaging for marketing in various quantity forms as per the need of end users such as aquarium fish exporters, importers, warehouses, retailers and like. The said effector composition reduces nitrates, nitrites, and ammonia level drastically in short time and close to 0% and retains them at low concentrations consistently. Further, the DAO, DAA, DA7 associated with transportation of aquatic animals can be bought down to 0%.

### REFERENCE TO RELATED APPLICATIONS

This application is a national stage application under 35 USC 371 of International Application No. PCT/IB2016/055689, filed Sep. 23, 2016, which claims the priority of India Application No. 201621019900, filed Jun. 10, 2016, the entire contents of which are incorporated herein by reference.

### FIELD OF THE INVENTION

The present invention generally relates to system & method to improve water quality by drastically bringing down nitrates, nitrites and ammonia close to 0% using microbial culture, more particularly, to systems and methods and bimolecular processes for improving productivity, health and life expectancy of fishes and other life forms in aquaculture industry, through the distribution chain.

### BACKGROUND OF THE INVENTION

Conventionally, in the aquarium industry, some of the major concerns are mortality at farming stage, during transportation and while at aquarium. The ecologists express concern over fish farming and trade considering the fact that the actual mortality values aren't revealed and may eventually lead to a major threat to the environment. At present, no solution is available to address major concerns associated with the aquarium industry from farm to end user and the distribution chain. Subsequently, a profitable, economical, ecologically viable, and productive solution is needed in the market around the globe to address said concerns.

There are a number of products available in the market. However, none of the presently available products is capable of bringing down DOA (Death on Arrival), DAA (Death after Arrival), DA7 (Death after 7 days) or mortality observed at aquariums to almost entirely to zero percent.

Another very important concern within the aquarium industry is nitrate, nitrite, and ammonia levels in the aquarium farm and/or tank. There are a number of products available in the market. However, none of the presently available products are capable of bringing down the nitrates, nitrites and ammonia close to zero percent and in short time.

Additionally, the presently available solutions don't possess characteristics to make offer said products as an inbuilt setup Aquaculture industry fish exporters, importers, and warehouses farms. Also, existing technologies suffer from multiple problems, including, lack of ability to get the DOA (Death on Arrival), DAA (Death after Arrival), and DA7 (Death after 7 days) lower than 0.5%.

There remains a need in the art to develop formulations that could bring about drastic reduction in nitrate, nitrite, and ammonia levels in a very short period of time and continue to curb the increase in concentrations of nitrates, nitrites and ammonia and address the mortality levels associated with Aquaculture industry to drastically low levels.

Patent number CN 101705202 A by He Jin et al titled, “Pseudomonas stutzeri YZN-001 for removing ammoniacal nitrogen, nitrate nitrogen and nitrous nitrogen in water body and application” discloses the microorganism used in the removal of water ammonia nitrogen, nitrate and nitrite. The limitation is it is used for sewage treatment and that to most important is there is limitation considering efficiency level of working.

Patent number CN CN103966138 A by Qiu Trick, Beijing Agricultural Biotechnology Research Center titled, “Paracoccus bengalensis N74-1 and application thereof in aquaculture water purification” discloses application of the strain N74-1 in aquaculture water purification.

Considering all above state-of-the-art, there is no any system and method available which suggest the effective way of denitrification and other mechanisms by which death of the fishes and other life forms in aquaculture industry can be brought down lower than 0.5%.

Accordingly, there remains a need in the aquarium industry to develop system and methods that provides end to end solution starting from farming stage, through the distribution chain to aquarium or retailers, end users which take care of life forms in aquaculture industry with better survival rates and with no mortality and good health in tanks, making aquaculture farming, trading and its distribution a more economical and profitable process. Thus, addressing the ecological concerns too.

### OBJECT OF THE INVENTION

The principal object of the embodiments herein is to provide efficient and cost effective effector composition, for improving productivity, health and life expectancy of a plurality of organisms in aquaculture industry, includes at least one microorganism species, a nutritive component and other supportive additives.

It is an object of the invention to provide the effector composition which reduces nitrates, nitrites, and ammonia level in the system virtually to 0% and maintains them at low levels consistently and gets the DOA (Death on Arrival), DAA (Death After Arrival). DA7 (Death after 7 days of Arrival) to virtually to 0%.

It is an object of the invention to reduce or avoid the mortality associated with stress and starvation very efficiently unlike any of the currently available products.

It is further an object of the invention to tackle the mortality issue observed in aquaculture industry, starting from farming stage to end user, during transportation and packaging to make supply chain and logistics related to aquaculture very profitable.

It is another object of the invention that the addition of effector formulation requires less amount of water during transportation, thus provide method & system for easy transportation of the aquaculture-fishes and other life forms.

It is further an object of the invention to provide effector composition which requires easy procedures for scaling up in order to cater from the farming stage, through the distribution chain to end user.

It is further an object of the invention that it makes the aquaculture very efficient in acclimatization, reducing the time of adjustment of the fishes and other life forms in aquaculture industry, in new environment.

It is another object of the invention that the appetite of the different types of fishes increases with the addition of said effector composition and showing signs of good health.

It is another object of the invention that addition of the said effector composition improves the colour and brightness of fishes and thus overall appearance.

It is another object of the invention that its use ensures healthy environment in the fish tank, farms, ponds and through the distribution chain.

It is another object of the invention that its use ensures clean water in aquarium systems starting from farms to ponds to tanks, with added benefit to aqua-plants and/or aqua vegetation.

It is another object of the invention that it will provide method & system which will reduce the maintenance of the fish tank and or aquarium and also at various stages starting from the farming stage through the distribution chain till the end users.

It is further an object of the invention to provide an effector composition wherein said microorganism species is a gram negative bacilli, which grows in medium selective for Pseudomonas species.

It is further an object of the invention to provide the said effector formulation as an integral part of setup in aquaculture industry such as aquarium fish exporters, importers, warehouses, end users or retailers.

It is further an object of the invention to address ecological concerns along with productivity and sustainable trade.

It is further an object of the invention to provide this effector formulation as a manufactured product supplied in suitable packaging to cater the need of the users such as exporters, importers, warehouses, retailers, end users.

### SUMMARY OF THE INVENTION

Accordingly, the embodiments herein provide efficient and cost effective, effector composition, system and method for improving productivity, health and life expectancy of aquaculture including plurality of organisms, and the said effector composition includes at least one microorganism species, a nutritive component and other supplements. In an embodiment the effector composition reduces nitrates, nitrites, and ammonia level in the system virtually to 0% and maintains them at low levels consistently and gets the DOA (Death on Arrival), DAA (Death After Arrival), DA7 (Death after 7 days of Arrival) virtually to 0%. In an embodiment effector composition help in reducing mortality associated with stress and starvation very efficiently unlike any of the currently available products. In an embodiment, the effector composition tackles the mortality issue observed in aquaculture industry through the distribution chain and aquariums, during transportation and packaging by reducing the nitrates, nitrites and ammonia buildup drastically. In an embodiment, effector composition thus ensures method & system for easy transportation of the fishes. In an embodiment, effector composition is easily scalable as per need of the customer or end user. In an embodiment, an effector composition contains one or more microorganism species, gram negative bacilli, which grows in medium selective for Pseudomonas species. In an embodiment, effector composition ensures healthy environment in the aquaculture industry, starting from farming stage including plurality of organisms such as ornamental fishes, starting from farming stage, distribution chain, retailers and end user fish tanks and like with reduction in maintenance.

Before undertaking the detailed description of the invention below, it may be advantageous to set forth definitions of certain words and phrases used throughout this patent document: the terms “include” and “comprise,” as well as derivatives thereof, mean inclusion without limitation; the term “or,” is inclusive, meaning and/or, the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of structure, operation, and methodology of the present invention and the objects and advantages thereof, reference is now made to the following descriptions taken in conjunction with the accompanying drawings, wherein like numbers designate like objects, and in which:

FIG. 1 illustrates a schematic representation of a mini experiment, which is performed on Angelfish prior to shipment in order to determine the best formulation in terms of the concentration to be used and composition, according to an embodiment of the invention;

FIG. 2 illustrates a schematic representation of an angelfish pre-treated with the said effector composition, according to an embodiment of the invention;

FIG. 3 illustrates a schematic representation of nitrate levels in the tank on treatment with the invention, as determined using the Mars Fishcare (API Fishcare) Nitrate test kit, according to an embodiment of the invention;

FIG. 4 illustrates a schematic representation of goldfish with dense packing and despite stressful parameters subjected to as described herein below, DOA, DAA, DA7 were found to be 0%, according to an embodiment of the invention; and,

FIG. 5 illustrates a schematic representation of a clown fish with dense packing and despite stressful parameters subjected to as described herein below, DOA, DAA, DA7 were found to be 0%, according to an embodiment of the invention.

FIG. 6 illustrates a schematic representation of sea urchins with and despite stressful parameters subjected to as described herein below, DOA, DAA, DA7 were found to be 0%, according to an embodiment of the invention.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF THE INVENTION

In the following description, for the purposes of explanation and not limitation, specific details are set forth such as particular architectures, interfaces, techniques, etc. in order to provide a thorough understanding of the present invention. However, it will be apparent to those skilled in the art that the present invention may be practiced in other embodiments that depart from these specific details. That is, those skilled in the art will be able to devise various arrangements which, although not explicitly described or shown herein, embody the principles of the invention and are included within its spirit and scope. In some instances, detailed descriptions of well-known devices, objects, and methods are omitted so as not to obscure the description of the present invention with unnecessary detail. All statements herein reciting principles, aspects and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure

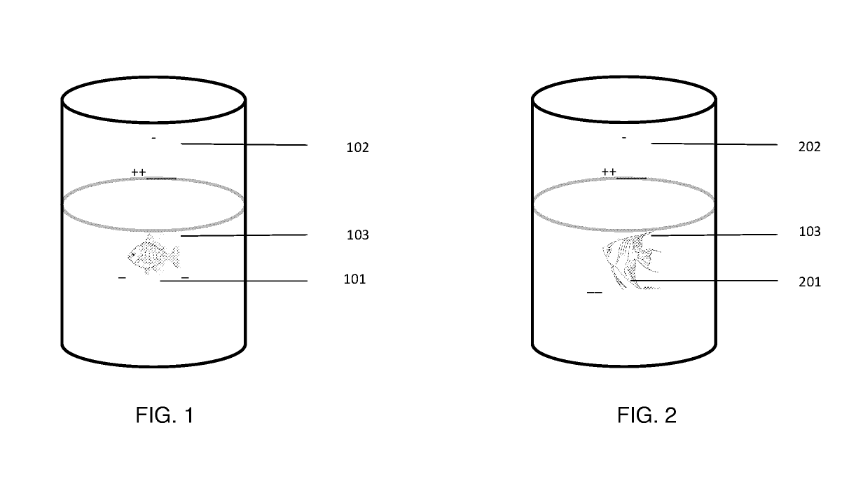

According to an embodiment of the present invention, FIG. 1 illustrates a schematic representation of a mini experiment, which is conducted on angelfish prior to shipment in order to determine the best formulation in terms of the concentration to be used and composition, according to an embodiment of the invention. This figure is of the fish 101, which did quite well and the product added to this container 102 was the chosen product formulation 103 for further studies.

According to an embodiment of the present invention, FIG. 2 illustrates a schematic representation an angelfish pre-treated 201 with the formulation 103 that were taken as control fishes all through the transportation experiment and were introduced in control tanks 202 after a journey in which they were subjected to various stress parameters. Not a single mortality observed and fishes immediately adjusted themselves to the environment thus ensuring quick acclimatization. Subsequently, it indicates that pre-treatment of fishes at farming stage may help the fishes to survive the stressful conditions, even if no product is added before or during shipment and DAA, DOA, DA7 is observed to be 0%. No mortality is observed in aquariums in which the present effector composition is added and has applications starting from farm level to ponds through the distribution chain to end users.

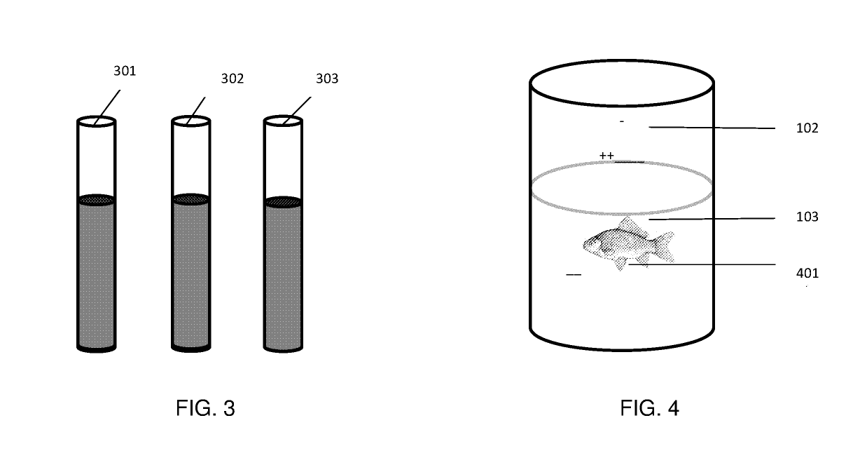

According to an embodiment of the present invention, FIG. 3 illustrates a schematic representation of nitrate levels in the tank determined using the Mars Fishcare (API Fishcare) Nitrate test kit, according to an embodiment of the invention. In Tube 1 301, aquarium tank untreated water is taken and it showed 150 ppm (red colour on comparison with Nitrate colour card) and in Tube 2 302, water is taken from untreated aquarium tank and water taken from water purifier in 1:1 proportion. The redness (on comparison with Nitrate colour card) reduced mildly indicating that dilution with fresh water didn't help to address nitrate levels. Tube 3 303 contains the aquarium tank+effector composition in appropriate proportions. Tube 3 shows the tank treated with the effector composition in appropriate proportions which enables drastic reduction in nitrate levels from 150 ppm to less than 5 ppm (yellow colour on comparison with Nitrate colour card) in a very short duration.

According to an embodiment of the present invention, FIG. 4 illustrates a schematic representation of a goldfish 401 with dense packing and despite stressful parameters they were subjected which are discussed in detail later, it shows DOA, DAA, DA7 were found to be 0%.

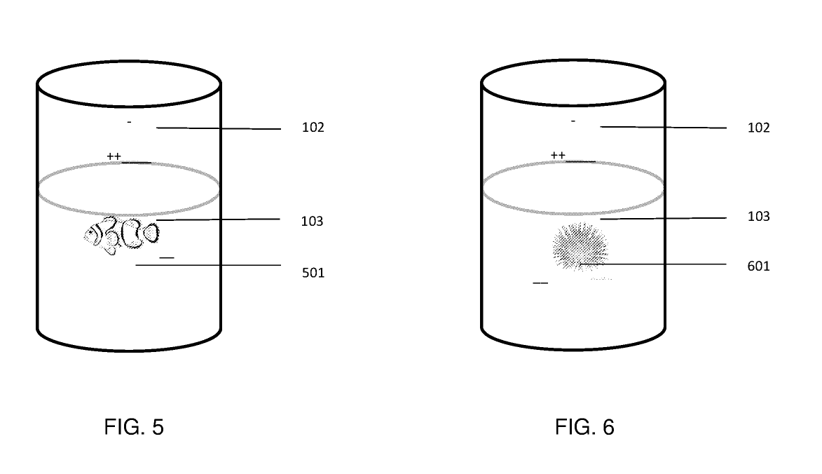

According to an embodiment of the present invention, FIG. 5 illustrates a schematic representation of a clown fish 501 with dense packing and despite stressful parameters they were subjected which are discussed in detail later, DOA, DAA, DA7 were found to be 0%.

According to an embodiment of the present invention, FIG. 6 illustrates a schematic representation of sea urchins 601 with and despite stressful parameters subjected to as described here in below, DOA, DAA, DA7 were found to be 0%, according to an embodiment of the invention.

In one embodiment, an effector composition for improving productivity, health and life expectancy of plurality of organisms in aquaculture industry such as ornamental fishes, includes at least one microorganism species and a nutritive component and other supportive additives. Particularly, the effector composition reduces nitrates, nitrites, and ammonia level and maintains them at low levels consistently, apart from the other features mentioned in the invention. The effector composition further includes about 2-10 ml of water while catering to a treatment of the multiple organisms in a 5 litre aquarium setup.

In preferred embodiment, one or more microorganism species are acquired from rhizospheres such as rice, wheat, grass, cotton, corn. Moreover, the microorganism species is selected from the roots and the surrounding soil using various microbial isolation techniques. Particularly, the microorganism species are gram negative bacilli, grow in medium selective for Pseudomonas species, and no growth to very scanty growth observed on Mac Conkey's agar. Further, the one or more microorganism species are Indole negative, Citrate negative, Urease negative, TSI slant—Non fermenter and MR—positive. The genetic sequence of the three bacterial isolates which have been part of the invention have been sequenced using NGS sequencing; whole Genome Sequencing.

The effector composition comprises in the relative amounts by weight of about 0.01 to 0.1 gm bacterial cells by weight, about 0.05 to 0.50 gm of any suitable nutritive components. Particularly, the above composition is for a 5 litres aquarium setup.

In one embodiment, nutritive component may include an additional source of all nutrients selected from a group comprising nutrient broth powder, vodka or sugars, easily available food such as those fed to ornamental fishes and aquatic animals or combinations of them or at least one carbon source, or organic and inorganic nutrients or like. A wide range of nutritive products could be formulated using different proportions, nutrients and additives refer to all those ingredients which help the microbes to multiply at a faster rate and help them establish themselves in the aquarium/farm setup. Most importantly, these nutrients and additives are chosen such that they cause no harm to the aquarium/farm environment.

In preferred embodiment, for applications related to bringing down nitrates, nitrites, ammonia in the tank, the following formulation is suggested as an example: 20-75 ml bacterial suspension+2.0 to 5.0 g of nutritive components to ensure optimal bacterial growth+50 to 150 ml water+50 to 150 ml of aquarium tank solution. The above mentioned formulation had immediate and drastic reduction in nitrates, nitrites and ammonia and getting the values close to 0 ppm. However, the present invention is not limited to the above proportion of the present effector composition and other similar proportions and permutations can be taken into account based upon the aquarium/farm size to address and reduce the nitrate, nitrite and ammonia levels in the aquarium tanks/farm.

In one embodiment, bacteria of the rhizosphere are present in a range of amount of not less than about 2×10¹¹ CFU/g to about 3×10¹¹ CFU/g of dry weight with respect to the total weight of the effector composition and the range is evaluated for a 5 litre capacity aquarium setup. The cfu/g could be calculated for larger aquarium systems and farms,

In one embodiment, the effector composition is selected from a suspension form or a dry form. Moreover, the effector composition is got to the log phase during the initial stages in aquarium/farm setup, following its addition. This enables the effector formulation to establish itself in the aquarium/farm setup.

In one embodiment, the multiple organisms are selected from a group including marine fishes, freshwater fishes, invertebrates, fragile, aggressive, colorful, sturdy, different size fishes having different behavioral pattern, larvae, fry, fingerlings and commercially important aquarium fishes.

In one embodiment, a system for improving productivity, health and life expectancy of multiple organisms includes an effector composition including at least one microorganism species, a nutritive component and other supportive additives. Particularly, the effector composition reduces nitrates, nitrites, and ammonia level drastically close to 0% and maintains them at consistently low levels, gets the DOA, DAA, DA7 virtually to 0% and other health benefits and features as described in this document. Generally, the multiple organisms are selected from a group including marine fishes, freshwater fishes, invertebrates, fragile, aggressive, colorful, sturdy, different size fishes having different behavioral pattern, larvae, fry, fingerlings, and commercially important aquarium fishes.

In one embodiment, the method further includes the steps of providing an effector composition including at least one microorganism species, mixing at least one microorganism species with sterile nutrient broth, and incubating on shaker at room temperature for about 48 hours to form a suspension. The nutritive components and time periods for incubation could vary. Particularly, the said effector composition reduces nitrates, nitrites, and ammonia level and retains them at low levels consistently and other features mentioned in this invention. The method further includes the step of dispersing the effector composition in a target environment for improving productivity, health and life expectancy of the multiple organisms in an aquarium.

Various methods as embodied by the present invention include multiple steps to address nitrates, nitrites and ammonia at a much quicker rate, such as, before adding the formulation, the skimmer needs to be turned off for few hours, and the formulation has to be added such that it gets into the rocks of the aquarium setup and appropriate, proportional quantity of the formulation is added along with its carbon or nutritional source such as media which enables microbes to multiply at faster rate and establish and ensuring that no harm is done to the aquarium/farm setup. In use, formulation must be added proportional to the size of the aquarium and/or aquarium tank and during addition, the aeration could be switched off for a while till the product gets evenly distributed in the tank and reaches the bottom rocks. In further use, the formulation introduced into the aquarium or farm, into the packaging bags for transportation enables the fishes to survive the most stressful, harsh environments and fishes treated with this product pass the different stress tests in contrast to the control in which no effector formulation was added to the fishes. However, the carbon source and the nutritive products or growth media for the effector composition could be of various kinds that are available in different proportions, in order to enable the effector composition to establish itself in the setup. Those of ordinary skill in the art will appreciate that the carbon source, one or more nutritive products for the bacterium and media could be of various kinds that are available in different proportions, with a view to enable the present formulation to establish itself in the setup it has been introduced, thereby observing its applications that are achieved much efficiently and quickly.

In one embodiment, the target environment is an aquarium tank aquarium farms, packages in which the fishes and other life forms of aquaculture industry are transported and the organisms are selected from a group including marine fishes, freshwater fishes, invertebrates, fragile, aggressive, colorful, sturdy, different size fishes having different behavioral pattern, larvae, fry, fingerlings, and commercially important aquarium fishes. The large varieties of fishes studied are found to be healthy, as would be in a natural habitat such as pond rather than a fish tank.

In use, the embodiments as disclosed herein may be applied during the farming stage and during subsequent transportation stages of fishes, from breeder to end-user (in other words through the distribution chain) and used at aquarium setup at importers, exporters, warehouses, retailers and end user's place. No mortality is observed when the present effector formulation is added. Fishes pre-treated with this product showed great potential in terms of survival in stressful conditions.

In one embodiment, the present effector composition works well at aquaculture industry, farm level and at their aquarium levels of different capacities such as 1000 litres, 150 litres, 10 litres, 5 litres, 4 litres and many such tanks of different size, shape and materials. Particularly, the effector composition has been tested at aquarium setup with artificial lights and aquariums with natural light source. The results were quite promising in both of the cases and not affected due to the light parameter.

In one embodiment, the aeration rate was chosen to be 2.5 L air/min as an instance. Generally, fishes die off if aeration is not provided for a few hours. The fishes could survive and be healthy even if the aeration system is put off for a day. The aeration rate could be varied in different setups and with the effector formulation added the aquarium fishes survive the stress of not having the aeration.

In preferred embodiment, the fishes could survive in the aquarium with no mortality and good health in tanks where water wasn't changed, such as for as long as 12 days.

In preferred embodiment, the effector composition enabled better appetite. The appetite of the different types of fishes stated above increased. In other words, before addition of the bacterium, the fishes weren't spontaneous in taking up the food. However, after the fish tank was treated with the effector composition, the fishes used to spontaneously take up the food and water was comparatively clearer than before. This is observed with addition of the same quantity of food, before and after addition of the effector composition. The addition of the effector composition led to fishes being more active and agile.

In preferred embodiment, on addition of the effector composition all the different types of fishes and other life forms of aquaculture industry had a behavior similar to what would have been while living in a natural habitat such as pond than a fish tank environment. Further, on addition of the effector composition, the brightness in fishes increased to an extent of 80%. Particularly, the fishes showed improvement in colour brightness within 30 minutes of the addition of the effector composition. In addition, growth and increase in size of the fishes is observed. With addition of the effector composition no incidence of aggressive fishes nipping the fins of other fishes occurred. The fishes were found healthy, as would be in natural habitat such as pond than a maintained fish tank.

In preferred embodiment, on addition of the effector composition, no mortality is observed. Before addition of the effector composition, there was at least 1 death per day.

In preferred embodiment, practically, in few days of addition of the effector composition, the water was clear and fishes are healthy, similar to if they were in a natural habitat such as the fishpond. The yellowish tinge in the experimental aqua plants was no longer there on addition of the effector composition. The leaves of all the plants appeared bright and green in colour.

In practice, the fishes that were treated with the effector composition were transferred back to the holding tank in which other fishes were kept ready for shipments. There seemed to be no stress with the fishes after staying in confined containers of only 5 litre compartments over 6 day period and no change in water. At the same time they seemed to be perfectly at home immediately upon introduction in the 150 litre holding tanks and started swimming well as if it was their native home. With the addition of the effector composition, the intervals at which the water needed to be changed reduced considerably.

Experiment Number One: The first experiment is conducted using the different microbial formulations and aim was to choose the best one based on the effectiveness observed for various features of the invention. Small Pterophyllum commonly called Angelfish are kept in 8 containers, one in each container. They fall under the family of Cichlidae. Small specimens approximately of the size of 1″ are selected considering their slightly fragile and delicate nature and also their characteristics to stay stable in small containers without fast movements, which enabled close observation of their skin, eyes, eating behavior etc. during this span of the experiment. In operation, 8 containers with 5 litres of freshwater are prepared with a single air-stone running 24×7 for a period of 6 days. Subsequently, eight Pterophyllum fishes are added one in each container from the same batch and of similar sizes and characteristics and observed in detail and reported.

The bacterial formulation is added in each 5 litre container and is about 5 ml. For purpose of comparison 5 rating are considered as a number that is neutral on the scale of 1-10. Particularly, the fishes were shifted from a big tank to the small containers and product formulation was added after about 30 minutes. The observations were recorded in tabular form for a week, on a daily basis. Table 1 below illustrates the observations at Day 1 of the experiment.

TABLE 1
Observations: Day 1

| Container | Scale (1-10) Worst to Best |
|---|---|
| Container 1 | Colour = 6, Movement = 5, Appetite = 5 |
| Container 2 | Colour = 6, Movement = 4, Appetite = 5 |
| Container 3 | Colour = 6, Movement = 5, Appetite = 5 |
| Container 4 | Colour = 6, Movement = 7, Appetite = 5 |
| Container 5 | Colour = 6, Movement = 7, Appetite = 5 |
| Container 6 | Colour = 6, Movement = 6, Appetite = 5 |
| Container 7 | Colour = 6, Movement = 6, Appetite = 5 |
| Container 8 | Colour = 6, Movement = 4, Appetite = 5 |
| (Control Tank with no | |
| Product addition) | |

Water Parameters: pH 7.8, Temperature = 28° C., Ammonia = 0.00 ppm

The summary of the observations are provided below, with focus on the container 3 which had the best formulation as determined based on various parameters. The details till the end of 6 days are as follows.

Day 1 observation: Water became hazy in all the tanks except in Control tank (#8).

Container 3—pH 7.8, Temperature=28 C, Ammonia=0.00 ppm, Colour=6, Movement=5, Appetite=5
For about 3 hours after adding the product fish movement was pretty slow. After this period fish started moving more comfortably. Fish was eating well. Colour slightly improved as if removing signs of stress. In this tank, the colour improvement was observed within 30 minutes of addition of the product.

Day 2 observation: All fishes seemed doing well including the one in the control tank. Water haziness had reduced considerably.

### Container 3:pH 7.8, Temperature=28 C, Ammonia=0.00 ppm Colour=7, Movement=7, Appetite=6

Colour was same like Day 1 of experiment. Fish movement slightly improved and so did the appetite

Day 3 Observation: It seems like there is not much difference between Day 3 and Day 2 of the testing. However it could also be said that all the fish have settled well within their containers or adapted better in their new containers.

Container 3—pH 7.8, Temperature=28 C, Ammonia=0.00 ppm, Colour=7, Movement=7, Appetite=6
Colour was same like Day 1 of experiment. Fish movement slightly improved and so did the appetite

Day 4 Observation: It seems there is very minor difference between Day 3 and Day 4 with the exception of Container 3. The fish in this specific container seems overall the best in terms of swimming behaviour and most appetite. Water in all the container has almost cleared out to ˜95% in comparison to Container 8.

Container 3:pH 7.8, Temperature=28 C, Ammonia=0.00 ppm, Colour=7, Movement=8, Appetite=7
Fish movement seemed improved compared to previous days. There was good appetite observed, in comparison to other fishes.

Day 5, 6 Observation: There seems to be not much difference in the observations between days or it could be the case of such slight gradual improvements that are hard to notice. Most of the results seems to be interpretable within first 2 to 4 days of adding the product with not much to make out in the subsequent days worth reporting.

Container 3: pH 7.8, Temperature=28 C, Ammonia=0.00 ppm, Colour=7, Movement=8, Appetite=8
Not much difference from previous days

Conclusion on Stage 1 of the experiment: Considering the overall tanks in totality and close observation over 6 day period is that container 3 fish seemed to be overall better. Ammonia buildup wasn't seen in any of these test containers, but observed in the Control tank. These fishes were now transferred back to the holding tank in which other fishes were kept ready for shipments. There seemed to be no stress with the fishes after staying in confined containers of only 5 Litre compartments over 6 day period and no change in water. At the same time they seemed to be perfectly at home immediately upon introduction in the 150 litre holding tanks and started swimming well as if it was their native home.

The experiment stated above is carried out on live specimens of freshwater fishes in order to select the best effector composition. The experimental setup as illustrated below is to study the effect of effector composition on nitrate, nirite, ammonia levels in tank and was carried out on saltwater tanks prior to actual shipment experiment. The experiment is conducted to determine the chemical composition and changes in saltwater tank. Table 2 below illustrates results of the tank parameters in which the quarantine marine fishes were added prior to shipment.

TABLE 2

| | Day 1 | Day 2 | Day 3 |
|---|---|---|---|
| Ammonia | 0.00 PPM | 0.00 PPM | 0.00 PPM |
| Nitrite | 0.00 PPM | 0.00 PPM | 0.00 PPM |
| Nitrate | 150 PPM | 20 PPM | <5 PPM |
| pH | 8.2 | 8.2 | 8.2 |
| Salinity | 1.023 sG | 1.023 sG | 1.023 sG |
| Temperature | 27° C. | 28° C. | 28.5° C. |

Day 1: Readings before addition of effector composition in a 150 litre sys-tem
Day 2: Readings after about 24 hours of addition of the effector composition to 150 litre system
Day 3: Readings after about 48 hours of addition of the effector composition to 150 litre system

About 2-5 ml of the effector composition is taken for a small pre-test before this experiment. Old marine tank water is added in a small container. The water before adding the product formulation tested ˜150-200 ppm nitrates, 0 ppm ammonia and 0 ppm nitrates. After addition of 2-5 ml of the effector composition to about 25 ml of the old saltwater in a flask, nitrates are tested and instantly became 0 ppm while other parameters such as ammonia and nitrites were totally unaffected. This is observed within a period of 10 minutes from addition of the effector composition.

The protocol followed to study the effect of effector composition on nitrates, nitrites and ammonia is as follows: The different parameters are determined on Day 1 at the beginning of the day before addition of the effector formulation. In operation, the effector composition is added at the end of Day 1. After addition of the effector composition the protein skimmers were switched off and all water circulation is stopped. An artificial carbon source in the form of Vodka or like is added. The tank, which is initially bare bottom and had no live sand or live rocks, is provided with highly porous lava rocks. The effector composition is added directly into the lava rocks and then the rocks are placed inside the tank. The skimmer is shut off for a period of 48 hours after adding the effector formulation. Following this, the marine fishes are added to this tank, skimmers are put on and aeration provided. After 48 hours, when the nitrate levels are determined, there wasn't any increase in nitrate levels. It is concluded that after adding the effector composition it is observed that there is dramatic reduction in nitrate. Also, following this, the fishes and invertebrates were introduced into the tank, aeration and skimmer put on, there wasn't increase in nitrates, nitrites and ammonia and the levels were maintained consistently at low levels. In the experiment, there was a buildup of nitrates and there was an urgent need to address these levels as had been requested. Hence, these steps of clearing out the tank, shutting aeration, skimmer off for the duration specified. The aim was help the microbe quickly establish itself in the aquarium. However, it isn't that the product would fail if skimmer or aeration aren't put off. Only while introducing the product, the skimmer may have to be put off for about 10 minutes for the product to get evenly distributed in the tank or like. The said effector composition has applications to address built up nitrates, nitrites, ammonia and in tanks and even where there is no buildup of these compounds, the said effector composition is required to maintain these nitrates, nitrites and ammonia at consistently low levels.

Experiment Number Two: To determine the effector composition's efficacy to reduce mortality associated with transportation due to stress and starvation. Many ornamental fishes and other life forms in aquaculture industry such as sea urchins are procured and put in the holding tanks. Greater variety of freshwater and marine fishes that are usually and more commonly available and which are traded more frequently and in huge numbers are selected. Different parameters were taken into consideration while selecting the fishes. For example, the fishes that are usually small, fragile, of economic importance, exquisite, popular were selected. Small fishes have a tendency to be more susceptible to damages in transportation, from water changes, from temperature fluctuations, from pH variations etc.

Moreover, fishes that are historically known to be fragile while being shipped are taken into account. Particularly, 7 tanks of 150 litres each for freshwater fish holding and 2 tanks of 150 litres, each for marine fishes were utilized for the experiment. The fishes are kept for a period of 12 days before the actual commencement of shipping. These fishes are fed properly, and the filtration systems kept working 24×7 in the form of power filter internal type as well as air stones to provide sufficient turbulence. This long period is primarily done to alleviate any stress from previous shipping at the time of receiving the fishes and to rule out any inherent diseases. All the fishes that are made available for shipping are disease free and in pretty good condition. Aquarium freshwater fishes such as Red Tail Cobra Guppies, Neon Tetras, Lemon Tetras, Endler Guppy, Hockeystick Tetras, Australian Rainbows (S), Australian Rainbows (M), Emperor Tetra, Galaxy Rasbora, African Malawi Cichlids, Oscar, Goldfish, Denisone, Angels, Silver Dollars were chosen for the study. Aquarium salt water fishes such as Butterfly fish (M), Nemo Clown Fish (S-M), Blue Ring Angel (XL), Lavender Tang (XL), Purpe Sea Urchins, Red Squirrel were chosen for this study.

The following experiment is conducted to determine the effectiveness of the said effect or composition while shipping the fishes. This study involved marine and freshwater tanks. In one embodiment, the fishes are tightly packed in far less than optimum conditions. For example, such as the packing density is more compared to usual standard. Further, the fishes are packed in smaller bags such that they have less space to swim. In such a case, it is expected that there will be about 10-20% mortality.

In some cases, the fish boxes were not stuffed with ice packs and the shipment go through higher stress especially comparing the ambient temperatures outside. No special care has been taken to insulate the boxes.

Packing procedures: High quality Styrofoam boxes are used. All fishes except saltwater fishes are packed in a group of 6-12 fish in each bag depending on the size. All bags are filled with ˜30% water and ˜70% oxygen. The oxygen used is high quality “Medical Oxygen” that is used for human consumption and not of Industrial grade. However, the bag sizes used for the present experiment were at least 1 size less than the optimum size required for packing either that quantity of fish and/or that size of the fish. All the boxes were packed and sealed and numbered.

Pre-shipping procedure: The said effector composition is added 30 minutes before packaging in 6 out of 7 Freshwater tanks and one tank served as control with no effector composition added. The said effector composition is added in 1 out of 2 saltwater tanks and the other saltwater tank serving as control. Particularly, in the case of the saltwater test tank, it had quite a lot of corals, the said effector composition was added in one area of the saltwater tank and skimmer and water circulation is kept off. Immediately after addition of effector composition, fishes in that portion of water is scooped out and packed. This is done primarily from preventing the product being mixed into the tank, to avoid loss of product (meant for this experiment) due to excessive corals in the tank. The angelfish from the first experiment (Stage 1) were in the control tank that had no said effector composition added i.e. the control tank were still marked for packing and shipping.

Observations: The effector composition is added in all the tanks 30 minutes prior to packaging such as to ensure that effector composition gets mixed well in the water and is also consumed by the fish in the tanks, except for the saltwater tank in which the effector formulation was added about 2 minutes before removing the water and fishes around it, into which the effector composition went, for reasons stated above. The fishes were transported from one city to another and then back to the same aquarium. In other words, the fishes were subjected to two freight journeys in a very short period of time.

During the day before receiving the shipped aquarium fish and invertebrates parcels, the water is replaced from 2 freshwater tanks and filled with new freshwater so as to remove the effector composition completely. These 2 tanks are re-designated as Control tanks. So in total there are 3 freshwater control tanks and 1 saltwater control tank.

Subsequently, fishes are added back in the tanks such that half the variety of each species went in tanks that had effector composition added the previous day and half of them went in control tank which either didn't have the effector formulation or the effector formulation is removed with complete exchange of water. It is observed that there were total of 8 fishes dead on DOA. Seven fishes died due to poor packing that resulted into leakage, and one due to aggression as Cichlids is packed together and all the death were observed in control tanks an no mortality in the tanks having effector composition. The experimental data described above cater to the aquarium level, farming level, pre-transportation stage, aquarium fish packaging stage, transportation stage, post-transportation and back to an aquarium or farm stages. The present invention effector formulation works as a great benefit while shipping live fishes used for aquarium trade.

It is observed that over 3 day period when the fishes were received, none of the fishes from the tank containing effector composition died. If there was stress associated with the shipment of the fishes, that too only 5 fishes died from control tank which didn't have the effector composition.

After the shipment it is observed that all fishes seem to have settled well and no mortality is observed. Generally, fishes in a normal setup of multiple tanks, which are used for trading, will observe certain percentage of deaths on a daily basis up to 2-3% on an everyday basis. However, by utilizing the present effector formulation the DAA has almost entirely come down to 0%.

Moreover, the fishes such as the fragile fishes, for example: Angelfish pretreated with the present invention before transportation were found to survive the stressful conditions mentioned above even if no effector formulation was added to their packaging. The pre-treatment itself gave the fishes the ability to survive in these harsh conditions and all through the Experiment 2, they were part of the Control tank setup. No mortality was observed in these fishes pre-treated with effector composition, despite being subjected to a combination of stressful conditions.

Further, the effect or composition introduced into the aquarium, and subsequently into the packaging bags for transportation enabled the fishes survive the most stressful, harsh environments and fishes treated with the effect or composition passed the different stress tests in contrast to the control bag of fishes in which no effect or composition was added. The term “stress” refers to the transportation conditions, temperature, handling of the fishes while transportation and/or during transit, duration of the fishes present in the packing system, ability to survive in packaging setup smaller than what is required which enables lesser water usage, and densely packed fishes far less than optimal conditions, i.e. packing density more compared to usual standard. The term “stress” also refers to the ability to stand all these stressful situations for more than 28 to 30 hours. Apart from these, “stress” also refers the ability to survive stress associated with sudden change in environment, bullying from other fishes, diseases and/or parasites, stress associated with nitrate, nitrite, ammonia build up in aquariums and the packages. This study covers stress associated at farm and aquarium levels and through the distribution chain and transportation and handling conditions.

The present effector composition calms the fishes during stressful transport conditions. The fishes that are treated with the effector composition are transferred back to the holding tank in which other fishes were kept ready for shipments. There seemed to be no stress with the fishes after staying in confined containers of only 5 Litre compartments over 6 day period and no change in water. At the same time they seemed to be perfectly at home immediately upon introduction in the 150 Litre holding tanks and started swimming well as if it was their native home.

In one of the experimental setup, the packaging box wasn't provided with ice packs and the shipment went through higher stressful conditions especially when taking into account the ambient temperatures outside. Also, no special care was taken to insulate the boxes with fishes and the fishes were subjected to high stress zone.

Fishes pre-treated with the effector composition showed great potential in terms of survival in stressful conditions. The fishes such as the fragile fishes pretreated with the effector composition before transportation are found to survive the stressful conditions mentioned above even if no effector composition is added to their packaging. The pre-treatment itself gave the fishes the ability to survive the harsh conditions.

In one embodiment, the effector composition got down the nitrate levels from 150 ppm to as less as <5 ppm in about 48 hours. No ammonia or nitrite accumulation is observed and all of them close to 0 ppm.

The present invention addresses nitrate, nitrite and ammonia levels by bringing them down drastically in aquariums with built up nitrates, nitrites and ammonia. Also, aquariums pre-treated with the effector composition didn't show any build up in nitrates, nitrites and ammonia. Practically, the effector formulation enables fishes to be compatible. Some fishes cause nipping of fins of other fishes. This isn't observed on addition of the effector formulation. The fishes are brighter in colour, better appetite, better movement or agility or vigour, calm in behaviour with reduced aggression and fishes were healthy and longevity is observed. Further, the effector formulation is beneficial to aqua plant. Water is clearer after few days of addition of this effector composition.

In one embodiment, the aeration rate is 2.5 L air/min, one of the instances. Usually, fishes die off if aeration is not provided for a few hours. However, with the addition of the effector composition, the fishes could survive and be healthy even if the aeration system was put off for a day. The fishes could survive in the aquarium with no mortality and good health in tanks where water wasn't changed for long such as 12 days, in other words, till the end of one of the analysis and observations.

In practice, the effector composition has been tested at aquarium setup with artificial lights and at aquarium set ups with natural light source. The results are quite promising in both of the cases and not affected due to this light parameter. In few days of addition of the effector composition, the water is clear and fishes are healthy, similar to if they were in a natural habitat such as the fish pond. The appetite of the different types of fishes stated above increased and led to a more healthy living. In other words, before addition of the effector formulation, the fishes weren't spontaneous in taking up the food. However, after the fish tank was treated with the effector composition, the fishes used to spontaneously take up the food and water was comparatively clearer than before, due to this. This is observed with addition of the same quantity of food, before and after addition of the effector composition. The addition of the effector composition led to fishes being more active and agile. Particularly, growth and increase in size of the fishes is observed. The yellowish tinge in the experimental aqua plants was no longer there on addition of the effector composition. The leaves of all the plants appeared bright green in colour.

Therefore, as may be seen, various embodiments as disclosed hereinabove provide a novel and innovative effector composition for enabling better life expectancy in aquaculture industry such as starting from ornamental fish farms through distribution chains to the ultimate customer. In use, various embodiments of the present invention disclose novel effector composition having application at farming stage wherein it shall be provided to the aquarium fish exporters, importers, warehouse owners as an integral part of their setup and if required as a manufactured product. Furthermore, various embodiments of the present invention may also be provided to the retailers and end users as a manufactured product available for sale. Additional advantages of present invention include healthier, more active, brighter fishes with a better appetite, whereby the fish colour is enhanced and greater longevity is observed. Also, reduced levels of aggression are displayed and within few days of addition of the effector composition, the water gets clearer.

Moreover, the present invention also addresses nitrate, nitrite and ammonia levels by bringing them down drastically in aquarium setup with had built up nitrates, nitrites and ammonia levels. Also, aquariums pre-treated with this effector formulation don't show any build up in nitrates, nitrites and ammonia. In use, pH levels are maintained and well within normal range found in aquarium setup, in large scale such as farming environment and/or aquarium tanks. In further use, fishes pre-treated with product embodied by various embodiments of present invention showed great potential in terms of survival in stressful conditions. Also, a notable feature is that fishes packed in bags in one size less than the optimal size did pass the stress test and DOA, DAA, DA7 was almost entirely 0%.

Consequently, various embodiments of the present invention also save water and freight charges, apart from shipment of live fishes with no mortality, thereby increasing productivity and addressing ecological concerns. The present invention discloses natural products with no adverse side effects, which are compatible with aqua-plants. In addition to above, various embodiments of present invention as disclosed hereinabove are aimed at providing an optimal healthy growth and development enhancement of fish colors, palatability, supply of micronutrients, breakdown of solid fish waste, reduced maintenance, increased water safety, and the like. In use, the present invention is aimed at providing a natural product that significantly improves productivity, health and life expectancy of Ornamental fishes and other life forms in aquaculture industry with Death On Arrival (DOA), Death After Arrival (DAA), Death after 7 days (DA7) brought down to almost entirely zero percent. The experiment is carried out and included commercially important aquarium fishes, sensitive & fragile fishes, both under the category of freshwater and marine fishes. Different varieties of fishes were subjected to multiple combinations of stressful factors as could be encountered in the industry, to determine on the efficiency of the present invention.

Moreover, the present invention addressed mortality associated with stress and starvation very efficiently unlike any of the currently available products. In use, the present invention discloses products containing aloe vera which help in reducing stress and heal damaged tissues by providing nutritional food pellets that take care of fish needs for as long as 14 days. Furthermore, present invention employs live bacteria products to consume sludge, thereby reducing aquarium maintenance. Depending on the location the effector composition can be used in a range of concentrations. Furthermore, the effector composition when used calms the fishes during stressful transport conditions. The effector composition enabled a clean water environment reducing the frequency at which water is changed.

The effector composition will be useful to aquarium fish exporters, importers, warehouses, end users or retailers. The effector composition will address ecological concerns along with productivity and sustainable trade.

1. A system and method, to improve productivity, health and Life expectancy of ornamental fishes and aquatic animals, uses an efficient and cost effective effector composition comprises of at least one microbial species, a nutritive component and other supportive additives.

2. The system and method as recited in claim 1, wherein the said microbial species is a gram negative bacilli, of which genome(s) have been sequenced using technologies such as Whole Genome Sequencing, which grows in medium selective for Pseudomonas species and which do not grow or grow very scantily on Mac Conkey agar and shows biochemical characteristics as indole negative, citrate negative, urease negative, TSI slant—Non fermenting and MR—positive and like.

3. The system and method as recited in claim 1, wherein the said bacterial species are present in a range of amount of not less than about 0.1×10¹¹ CFU/g to about 10×10¹¹ CFU/g of dry weight with respect to the total weight of said effector composition wherein said range is evaluated for a 5 litre capacity aquarium setup.

4. The system and method as recited in claim 1, wherein the microbial, enzymatic, biomolecular, and combined nutritive are present in the relative amounts by weight of about 0.01 gm to 0.5 gm bacterial cells by weight, about 0.01 gm to 1 gm of nutritive components, wherein said composition is for a 5 litre capacity aquarium setup.

5. The system and method as recited in claim 1, wherein the said microbial species is isolated from a rhizosphere such as rice, wheat, grass, cotton, corn and like.

6. The system and method as recited in claim 1, wherein said effector composition drastically reduces nitrates, nitrites, and ammonia level in a very short time and consistently maintains them at low levels such as about 150 ppm was bought down to as less as <5 ppm in about 48 hours and maintains at such low levels consistently and gets the DOA (Death on Arrival), DAA (Death After Arrival), DA7 (Death after 7 days of Arrival) virtually to 0%.

7. The system and method as recited in claim 1, wherein said effector composition reduces the mortality associated with starvation and various stress factors associated with the aquaculture industry and their practises, such as in the ornamental fish industry and trade.

8. The system and method as recited in claim 1, wherein said effector composition reduces the mortality virtually to 0%, in aquariums or like, throughout the distribution chain starting from farm to end users, during transportation and packaging or like.

9. The system and method as recited in claim 1, wherein said effector composition improves transportation system for ornamental fishes and aquatic animals by reducing the amount of water required during safe transportation and thus assuring efficient supply and logistics.

10. The system and method as recited in claim 1, wherein said effector composition ensures easy scaling up procedures, which enables application of various features of effector composition enlisted under claims, to be used in different scales starting from farm level, warehouses, through the distribution chain to the retailers and end user's aquariums.

11. The system and method as recited in claim 1, wherein the said effector composition, ensures reduction in the time of acclimatization of the aquatic animals in new environment.

12. The system and method as recited in claim 1, wherein the said effector composition, increases the appetite of the different types of aquatic animals that ensures the health of these animals.

13. The system and method as recited in claim 1, wherein the said effector composition, improves the colour and brightness of aquatic animals by almost 80% and thus improves overall appearance and adds aesthetic value.

14. The system and method as recited in claim 1, wherein the said effector composition, ensures healthy environment in the aquarium setup or like, including healthy behavioral pattern in the aquarium setup or like improved vigor, reduced levels of aggression, very active and agile and compatibility with aqua-plants.

15. The system and method as recited in claim 1, wherein the said effector composition, ensures long term clean water in aquarium without any added procedures along with added benefit to aqua-plants and/or aqua vegetation.

16. The system and method as recited in claim 1, wherein the said effector composition, reduces the maintenance of the fish tank and or aquarium such as reduction in the intervals at which the water needed to be changed and like.

17. The system and method as recited in claim 1, wherein the nutritive component in the said effector composition includes different proportions of nutrient broth powder, vodka, sugars, easily available food such as those fed to aquatic animals, inorganic and organic nutritive compounds, combinations of them or like or at least one carbon source required for optimal bacterial growth, in-order to establish itself in given environment.

18. The system and method as recited in claim 1, wherein the supportive additives in the said effector composition can ensure proper packaging of the formulation and the viability and efficiency of product.

19. The system and method as recited in claim 1, wherein the aquatic animals can be ornamental fishes, marine fishes, freshwater fishes, invertebrates, aqua plants including fragile, aggressive, colorful, sturdy, different size fishes having different behavioral pattern, larvae, fry, fingerlings and commercially important aquarium fishes or like.

20. The system and method as recited in claim 1, wherein the said effector composition can be made available in various forms, quantities, packages to aquaculture industry including aquarium fish exporters, importers, warehouses, retailers and any other end users as a manufactured product and/or as integral setup of their unit.

21. The system and method as recited in claim 1, wherein the said effector composition may be selected from a suspension or a dry form or like and wherein the said effector composition is preferred to be in a log phase during the initial stages so that after addition into the requisite water body the microbes will multiply and establish themselves easily in short time.

22. The system and method as recited in claim 1, wherein the said effector composition can be used at any level as aquarium, farming, pre-transportation stage, aquarium fish packaging stage, transportation stage, post-transportation and back to an aquarium or farm stages and works as a great benefit while shipping live aquatic animals like ornamental fishes, used for aquarium trade.

23. The system and method as recited in claim 1, wherein pretreatment of aquatic animals including ornamental fishes with the said effector composition greatly improves potential in terms of survival of aquatic animals including ornamental fishes in stressful conditions even if the said effector composition is not added to their packaging.

24. The system and method as recited in claim 1, wherein pretreatment of aquatic animals including ornamental fishes with the said effector composition greatly improves potential in terms of survival of aquatic animals including ornamental fishes in a stressful conditions such as reduction in the time for which the aeration system in aquarium set up is generally kept on thus saving the electrical energy and reducing the maintenance cost.

25. The system and method as recited in claim 1, wherein the said effector composition have no any untoward side effects as a product is derived from nature.

26. The system and method as recited in claim 1, wherein the said effector composition also addresses ecological concerns related to aquatic animals trade like ornamental fishes along with improving the productivity by providing sustainable solution.
